(19) United States
(12) Patent Application Publication
(10) Pub. No.: US 2025/0288548 A1
(43) Pub. Date: Sep. 18, 2025

(54) INTRANASAL BACLOFEN

(71) Applicant: THE REGENTS OF THE UNIVERSITY OF CALIFORNIA, Oakland, CA (US)

(72) Inventor: Andrew SKALSKY, La Jolla, CA (US)

(21) Appl. No.: 18/859,920

(22) PCT Filed: May 2, 2023
(86) PCT No.: PCT/US2023/020741

§ 371 Date: Oct. 24, 2024

Related U.S. Application Data
(60) Provisional application No. 63/337,585, filed on May 2, 2022.

(51) Int. Cl. A61K 31/197; A61K 9/00; A61M 15/08

(52) U.S. Cl. CPC A61K 31/197; A61K 9/0043; A61M 15/08

(57) ABSTRACT

An intranasal baclofen formulation, an intranasal delivery device, and methods for the treatment of dystonia and muscle hypertonicity are provided.

### RELATED APPLICATIONS

This application claims priority from U.S. Provisional Application Ser. No. 63/337,585 filed on May 2, 2022, which is incorporated herein by reference in its entirety.

### FIELD OF THE INVENTION

This application relates to, among other things, formulations containing baclofen that are stable in, compatible with, and deliverable via an intranasal administration device.

### BACKGROUND

Baclofen is a skeletal muscle relaxant and antispastic agent. Baclofen is a structural analog of the inhibitory neurotransmitter gamma-aminobutyric acid (GABA) and may exert its effects by stimulation of the GABAB receptor subtype.

LIORESAL® Intrathecal (baclofen injection) has been developed for chronic intrathecal infusion for the management of severe spasticity. Baclofen can be administered orally, but when injected directly into the intrathecal space of a patient, therapeutically effective intrathecal concentrations are achieved with resultant plasma concentrations 100 times less than those occurring with oral administration. Baclofen injections (LIORESAL® Intrathecal, Medtronic, Inc.) are therefore commonly administered intrathecally to manage severe spasticity of spinal cord origin. Presently, baclofen is commercially available for intrathecal injection, as a 0.05 mg/mL solution, a 0.5 mg/mL solution or a 2 mg/mL solution having a pH of 5 to 7 in the following preservative-free formula (LIORESAL® Intrathecal): baclofen (0.05 mg, 0.5 mg or 2 mg); sodium chloride (9 mg); water for injection q.s. 1 mL.

The 0.5 mg/mL and 2 mg/mL concentrations have proven to be very effective for delivery via an implantable infusion device, such as Medtronic, Inc.'s SYNCHROMED II® infusion device, for a number of patients suffering from severe spasticity. However, such a device requires a surgical procedure to implant the infusion device and catheter which is associated with a significant infection and complication rates as well as requirement for repeat implantation regularly due to limitations in battery life of the implanted device.

In fact, Haranhalli et al. indicated that approximately 20-30% of patients with intrathecal baclofen pumps experience some type of complication. On average, a patient will require an average of 3.5 procedures with a total risk of infection of 14.6% and a total risk of pump and catheter problems of 19.7%.

Complications can be categorized as both mechanical and/or infection-related. Common mechanical complications include pump failure (battery failure or pump stalling), catheter difficulties (kinking, breaking, fracturing), CSF leak at the site of catheter insertion, and formations of adhesions around the catheter site. Common types of infections include wound infections, pump or catheter infections, or, in rare cases, meningitis. Having an intrathecal baclofen pump requires patients to attend regular follow-up appointments where medical providers will monitor the dose, refill the pump, and surgically replace the pump when the battery is near expiration.

The surgical risk and complications limit a large number of patients from accessing intrathecal baclofen therapy even though it is more effective than enteral medication. With only the enteral and intrathecal drug delivery routes available, a large unmet need exists for optimization of the treatment of hypertonicity. Therefore, there is a clinical need to provide a non-invasive and cost-effective spasticity treatment having minimal side effects.

### SUMMARY

The present teachings provide a method of treating a patient with a disorder that may be treatable with a baclofen, the method comprising: administering to one or more nasal mucosal membranes of a patient a pharmaceutically acceptable solution for nasal administration consisting of a baclofen in a saline solution. In various embodiments, the disorder can be one or a combination of muscle hypertonicity, spasticity, and dystonia. In a further embodiment, the saline solution further comprises an additive selected from the group consisting of citric acid, cyclodextrin, and hypromellose. In yet another embodiment, the pharmaceutical solution is in a pharmaceutically acceptable spray formulation having volume from about 10 μL to about 200 μL. In another embodiment, the administration of the pharmaceutical solution comprises spraying at least a portion of the therapeutically effective amount of the baclofen into at least one nostril. In another embodiment, the administration of the pharmaceutical solution comprises spraying at least a portion of the therapeutically effective amount of the baclofen into each nostril. In another embodiment, the administration of the pharmaceutical solution comprises spraying a first quantity of the pharmaceutical solution into the first nostril, spraying a second quantity of the pharmaceutical solution into a second nostril, and optionally after a pre-selected time delay, spraying a third quantity of the pharmaceutical solution into the first nostril. In various aspects, the administration further includes, optionally after a pre-selected time delay, administering at least a fourth quantity of the pharmaceutical solution to the second nostril. In yet another embodiment, nasal administration of the pharmaceutical solution begins at any time before or after onset of symptoms of a disorder which may be treatable with the pharmaceutical solution. In another embodiment, the treatment achieves bioavailability that is from about 80-125% of that achieved with the same baclofen administered intrathecally.

Further teachings provide a pharmaceutical solution for nasal administration consisting of: (a) a baclofen or its pharmaceutically acceptable salt; and (b) one or more of citric acid, cyclodextrin, and hypromellose, or any combinations thereof, in an amount from about 30% to about 95% (w/w); in a pharmaceutically acceptable formulation for administration to one or more nasal mucosal membranes of a patient. In various embodiments, the baclofen is present in the pharmaceutical solution in a concentration from about 2.0 mg/mL to about 10.0 mg/mL.

Yet further teachings provide a device for delivery of a pharmaceutical solution, the device comprising a pharmaceutical solution of a baclofen or its pharmaceutically acceptable salt; and one or more of citric acid, cyclodextrin, and hypromellose, or any combinations thereof, in an amount from about 30% to about 95% (w/w); in a pharmaceutically acceptable formulation, wherein the pharmaceutical solution is stored in a cartridge; an atomizer, wherein the atomizer receives the baclofen formulation from the cartridge and delivers the baclofen formulation to a patient's nasal cavity; an optional dose adjustment control, wherein the dose adjustment control determines the amount of the baclofen formulation to be delivered; and a spray control, wherein the spray control triggers the baclofen formulation to be delivered to the patient's nasal cavity.

In one aspect of the invention, a method of treating a patient with a disorder that may be treatable with a baclofen includes administering to one or more nasal mucosal membranes of a patient a pharmaceutically acceptable solution for nasal administration consisting of a baclofen in a saline solution. The disorder may be selected from the group consisting of muscle hypertonicity, spasticity, and dystonia. In some embodiments, the saline solution further includes an additive selected from the group consisting of citric acid, cyclodextrin, and hypromellose. The pharmaceutical solution may be in a pharmaceutically acceptable spray formulation having volume from about 10 μL to about 200 μL. Administration of the pharmaceutical solution may include spraying at least a portion of the therapeutically effective amount of the baclofen into at least one nostril or spraying at least a portion of the therapeutically effective amount of the baclofen into each nostril. Administration of the pharmaceutical solution may include spraying a first quantity of the pharmaceutical solution into the first nostril, spraying a second quantity of the pharmaceutical solution into a second nostril, and optionally after a pre-selected time delay, spraying a third quantity of the pharmaceutical solution into the first nostril. Optionally, after a pre-selected time delay, at least a fourth quantity of the pharmaceutical solution may be administered to the second nostril. Nasal administration of the pharmaceutical solution may begin at any time before or after onset of symptoms of a disorder which may be treatable with the pharmaceutical solution. The treatment achieves bioavailability that is from about 80-125% of that achieved with the same baclofen administered intrathecally.

In another aspect of the invention, a pharmaceutical solution for nasal administration includes (a) a baclofen or its pharmaceutically acceptable salt; and (b) one or more of citric acid, cyclodextrin, and hypromellose, or any combinations thereof, in an amount from about 30% to about 95% (w/w); in a pharmaceutically acceptable formulation for administration to one or more nasal mucosal membranes of a patient. The baclofen may be present in the pharmaceutical solution in a concentration from about 2.0 mg/mL to about 10.0 mg/mL.

These and other features, aspects and advantages of the present teachings will become better understood with reference to the following description, examples and appended claims.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 represents the summarized results of a study of about 40 patients. Grey bars represent patients treated with nasal baclofen, and white bars represent placebo. Bars to the left (negative) represent reduced tone, and right (positive) is worse tone. The middle line is no change. Statistically significant results are provided with error bars in this figure.

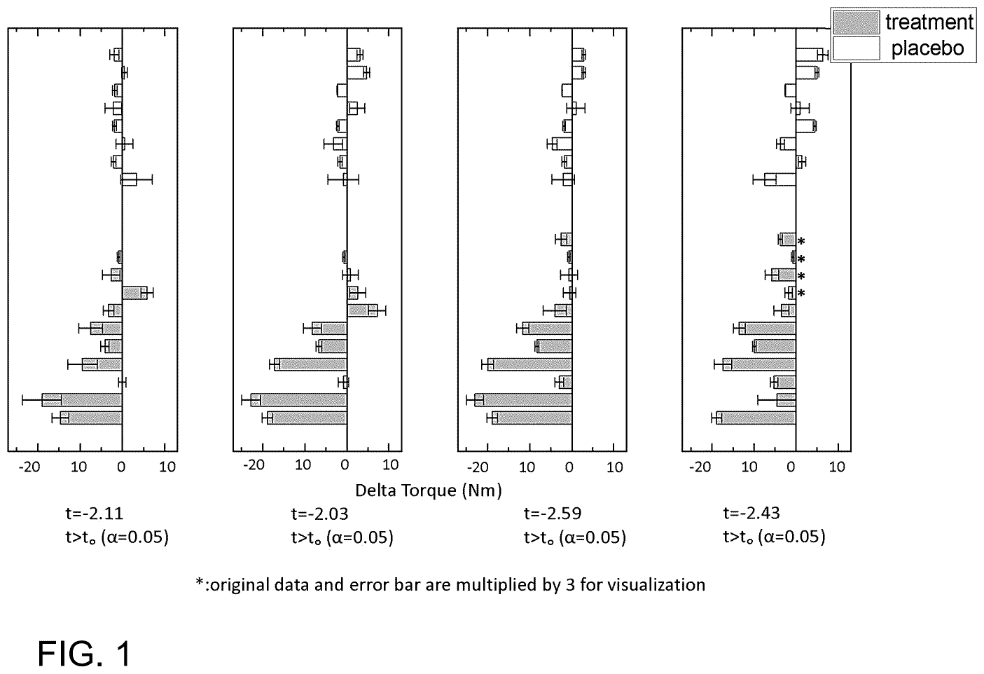

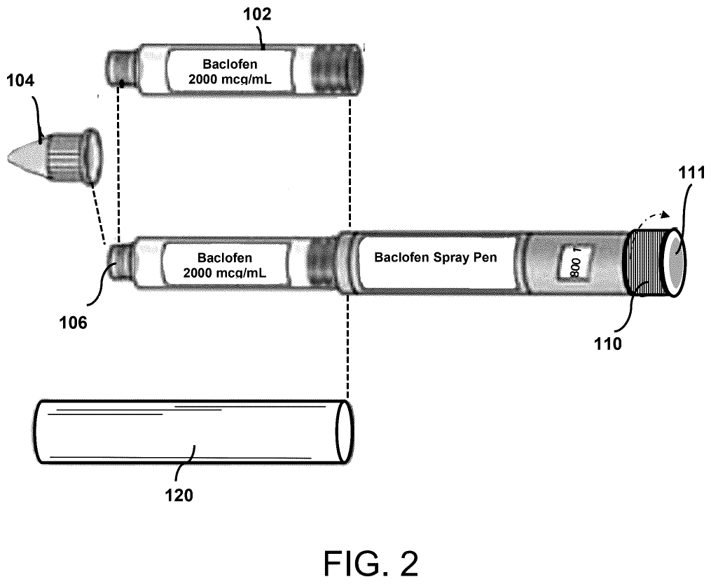

The figure shows the delta (change) in measured torque response from baseline for each patient visit. The first bar chart from left to right is baseline compared to visit 2 low dose (250 mcg). The second bar chart from left to right is baseline compared to visit 3 moderate dose (1000 mcg). The third bar chart from left to right is baseline compared to visit 4 high dose (2000 mcg). The fourth bar chart from left to right is baseline compared to visit 5 weaned off back to baseline (providing some residual effect). The p-values for the third panel, which represents the high dose are: Treatment p-value 0.004959, and Control p-value 0.232.

FIG. 2 is a depiction of an intranasal baclofen delivery device 101. The delivery device comprises a baclofen cartridge 102 (shown in FIG. 2 in an upper position when unattached) that can be fitted onto spray pen housing 103. An atomizer 104 can be fitted onto the baclofen cartridge 102 at the atomizer attachment point 106. Optionally, the dosage of baclofen administered to a patient can be controlled by turning a dose adjustment dial 110. The baclofen dosage is administered into a nostril of a patient using the spray button 111. Optionally, a cap 120 may be provided to enclose the baclofen cartridge and atomizer when attached to the spray pen housing.

### DETAILED DESCRIPTION OF EMBODIMENTS

Hypertonicity is a common symptom in individuals with central nervous system (CNS) lesions. Muscle hypertonicity can be of cerebral or spinal origin that can manifest as spasticity, dystonia, or rigidity and frequently a combination is present. Many conditions lead to hypertonicity including cerebral palsy, acquired brain injury, metabolic disorders, leukodystrophies, strokes, multiple sclerosis, hydrocephalus, or spinal cord injury. Patients with hypertonicity are unable to produce smooth and fluid limb movements due to the imbalance of signals from the brain and spinal cord and can be with or without spasticity. Severe spasticity is associated with pain, sleep disorders, feeding problems, and difficulty with activities of daily living such as positioning, transfers, dressing, and hygiene.

Treatment of hypertonicity involves a comprehensive evaluation by a large multidisciplinary team of professionals to develop a treatment plan unique to the child and overall goals of the family. Available treatments for hypertonicity include physical therapy (e.g., constraint-induced therapy), enteral medications, injections of botulinum toxin, intrathecal baclofen (ITB), and dorsal rhizotomy.

Baclofen, a γ-aminobutyric acid receptor b (GABA_{B}) agonist, was first reported as a treatment for spasticity by Hudgson et al. As a widely used medication that has been in the market since its FDA approval in 1977, baclofen is recommended to treat severe spasticity or hypertonicity.

Baclofen was originally administered enterally and is readily absorbed from the gastrointestinal tract quite quickly, however, only a small proportion of the dose crosses the blood-brain barrier (BBB) and ends up reaching its sites of action in the central nervous system. Enteral baclofen doses ranging from 30 to 90 mg are associated with plasma levels of 0.05-0.65 mcg/mL and CSF levels of <0.012-0.096 mcg/mL.

The effects of systemic baclofen to affect the central nervous system CNS are limited by the inability of baclofen to readily cross the blood brain barrier. Baclofen is dependent on amino acid transporters to cross into the cerebrospinal fluid.

Penn et al. introduced intrathecal baclofen (ITB) which directly bypasses the blood brain barrier as a treatment for spasticity. When injected directly into the intrathecal space of a patient, therapeutically effective intrathecal concentrations of baclofen are achieved with resultant plasma concentrations 100 times less than those occurring with enteral administration. Administering baclofen intrathecally increases the concentration of baclofen present in the CSF in adults: an intrathecal dose of 400 micrograms was associated with a concentration of nearly 400 micrograms/milliliter in the CSF and low concentrations in the serum.

In 1996, ITB administered by an implanted pump was approved by the US Food and Drug Administration for the treatment of spasticity. ITB has been clinically shown to be more effective in relieving spasticity than enteral baclofen. It is available for patients who have not seen an adequate reduction in their hypertonicity, or who have experienced unsustainable adverse side effects from enteral baclofen. Using ITB allows for the dose of baclofen to be maximized and its peripheral side effects to be minimized.

ITB therapy involves a surgical procedure that implants a subcutaneous pump into the abdomen of the patient, in addition to implanting a catheter system into the spinal canal with the catheter tip within the cerebral spinal fluid (CSF). Direct access to the CSF results in a higher concentration of baclofen in the CNS, which also results in a smaller dosing regimen than what is required for systemic administration.

However, ITB therapy requires a surgical procedure to implant the infusion device and catheter which is associated with a significant infection and complication rates as well as requirement for repeat implantation regularly due to limitations in battery life of the implanted device, an easier to administer form of baclofen to achieve with similar efficacy to intrathecal baclofen but ease of administration of enteral baclofen is desired.

ITB therapy is the most effective treatment for severe spasticity and dystonia, however, the complication rate associated with it is relatively high as well as requirement for repeat implantation regularly due to limitations in battery life of the implanted device.

It is necessary for patients to have access to specialized medical care, and the patient and caregiver must be capable of adhering to the required maintenance of the pump. Reluctance of caregivers is a barrier to care for patients. In very small or thin patients, the size of the pump can be problematic. Pump refill may not be possible inpatients that are extremely overweight, this is compound by the fact that weight gain can occur after the pump is implanted. Despite these limitations, studies have suggested that most patients with an ITB pump and their caregivers were satisfied with the outcomes resulting from this treatment.

### Blood Brain Barrier Traverse

The blood brain barrier (BBB) is the main regulator of drug transport to the CNS by a strictly controlled and dynamic process via the specific transport pathways. From a clinical perspective, the problem is twofold: neuropharmaceuticals may not effectively penetrate the BBB well as is the case with enteral baclofen, and neuropharmaceuticals in the systemic circulation undergo first pass metabolism which can result in active metabolites with adverse effects which is also the case with enteral baclofen.

A noninvasive delivery, specifically nasal administration, has several advantages over traditional enteral medication as this targets the delivered dose absorption higher than enteral route—translating to less dosage and less susceptibility to complications of overdosing. Furthermore, nasal delivery avoids complications from invasive surgery which is required for the intrathecal administration of baclofen.

Nasal administration bypasses first-pass metabolism that is observed in enteral delivery and the lung and nasal cavity have a low drug metabolizing environment. The nasal route for delivery of drugs to the brain via the olfactory region is an optional solution to improve penetration across the BBB.

In contrast to invasive strategies for circumventing the BBB such as an implanted pump, the nasal pathway can be exploited for the non-invasive delivery of drugs to the CNS. The nasal epithelial surface area is small, about 150 cm², and consists of respiratory and olfactory regions. Although the olfactory system possesses elaborate epithelial, endothelial and glial barriers, due to its special anatomical localization it provides a direct access to brain. Intranasal delivery gives efficacy of intrathecal delivery but convenience of enteral administration without the need for a surgical procedure or an implanted pump.

The mechanism of nasal absorption of baclofen tested in animals suggest that the rate of absorption in the nasal mucosa is relatively large occurring via passive diffusion and via a carrier-mediated mechanism responsible in transporting baclofen across the BBB.

The nasal mucosa efficiently absorbs both enantiomers of baclofen without specificity. The observed concentration independent transport and the absence of polarized flux in the excised nasal tissues suggest that baclofen is primarily absorbed via simple passive diffusion across the bovine nasal tissues.

Nasal administration of baclofen can result in faster presentation of the drug to the brain due to the close proximity of the nasal membranes and the brain. A spastic patient, for example, can therefore see relief faster when compared with oral and/or intrathecal administration.

### Intranasal Baclofen and Pharmaceutical Solutions for Nasal Administration

Baclofen as used herein refers to 4-amino-3-(p-chlorophenyl) butyric acid, enantiomers, racemic mixtures, polymorphs, salts, solvates, esters, or hydrates thereof. Baclofen is described by the following structural formula:

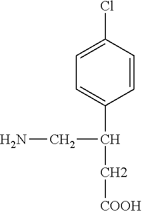 [SMILES: NCC(CC(=O)O)c1ccc(Cl)cc1]

Baclofen includes R-baclofen (D baclofen), S-baclofen (L-baclofen), or their mixtures including the racemate. The racemate refers to a mixture of R and S-baclofen (DL-baclofen) in equal proportions.

The phrase “pharmaceutically acceptable salt(s)”, as used herein, means those salts of baclofen that are safe and effective for use in mammals and that possess the desired biological activity. Salts of baclofen include salts of acidic or basic groups present compounds of the application. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, or pamoate salts. Certain compounds of the invention can form pharmaceutically acceptable salts with various amino acids. Suitable base salts include, but are not limited to, aluminum, calcium, lithium, magnesium, potassium, sodium, zinc, or diethanolamine salts. Potassium salts include potassium chloride, potassium bicarbonate, potassium phosphate, gluconate, potassium citrate, or the like.

It should be recognized by those of skill in the art that baclofen and its pharmaceutically effective salts have low solubility and can be modified in solutions for delivery intranasally at higher concentrations via the nasal route, faster attainment of therapeutic levels of baclofen in the CNS, avoidance of the liver portal vein and concomitant avoidance of first pass effects and/or faster presentation of baclofen drug to the brain. Pires et al. describe several modifications, including the provision of carrier systems, that can be used to enhance the solubility of baclofen and hence provide for more concentrated baclofen formulations. See Pires, P. C., et al., Strategies to Improve Drug Strength in Nasal Preparations for Brain Delivery of Low Aqueous Solubility Drugs. Pharmaceutics 2022, 14, 588 (available at DOI handle “10.3390/pharmaceutics14030588”), incorporated herein by reference in its entirety.

For example, some baclofen salts are slightly soluble in water such that a therapeutically effective amount cannot be dissolved in a volume of aqueous solvent that is amenable to application to a mucosal membrane. By use of a carrier system, the present invention allows baclofen to be administered to one or more mucosal membranes, including to nasal mucosal membranes. This can allow one to administer the drug without hospitalization or unnecessary discomfort. Additionally, in some embodiments of the present invention, such as nasal administration, the digestive system largely may be bypassed. This latter improvement can yield improved bioavailability, faster attainment of therapeutic levels of baclofen in the CNS and/or blood plasma, avoidance of the liver portal vein, and/or concomitant avoidance of first pass effects.

As provided in U.S. Pat. No. 9,180,108, it has been found that the solubility of baclofen is increased in solutions comprising sulfate or phosphate. It has been described that concentrations greater than 2 mg/mL, such as greater than 4 mg/mL, greater than 5 mg/mL, greater than 6 mg/mL, or greater than 7 mg/mL baclofen can be dissolved in solutions containing phosphate, sulfate, potassium, or magnesium.

The pharmaceutical solution may have any suitable ionic strength. The pharmaceutical solution may include a salt, such as NaCl or KCl, to maintain ionic strength. In various embodiments, the combined ionic strength of the pharmaceutical solution contributed by the various components of the formulation, such as baclofen, and one or more salts is the equivalent of the ionic strength of between about 0.1 M and 0.2 M NaCl or KCl, or about 0.15 M NaCl or KCl.

Preferably, the pharmaceutical solution has a pH of between 5.0 and 7.5, such as between 5.5 and 6.5, or about 6.0. For example, the pH can be about 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5.

In some embodiments, the present invention describes the use of stable aqueous baclofen solutions at concentrations greater than 2.0 mg/mL. In particular, in some embodiments, the present invention provides the use of stable aqueous baclofen solutions having concentrations greater than about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9. 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 mg/mL.

In various embodiments, the pharmaceutical solution containing baclofen includes (i) baclofen in a concentration greater than 2 mg/mL; and (ii) optionally a sulfate or phosphate in a concentration of between 5 mM and 25 mM. The solution has an ionic strength equivalent of between 1.4M NaCl and 1.6 M NaCl. In some embodiments, the solution consists essentially of the baclofen, the sulfate or phosphate, sodium ion, and water. In embodiments, the solution consists essentially of the baclofen, the sulfate or phosphate, sodium ion, chloride ion (e.g., from NaCl), and water.

In some embodiments, the aqueous solvent is preferably water, a saline solution (which, as used herein, means a solution consisting of or consisting essentially of water and dissolved sodium chloride), a sulfate or phosphate solution (which, as used herein, means a solution consisting of or consisting essentially of water and dissolved sulfate or phosphate salt or acid), or a sulfate or phosphate saline solution (which, as used herein, means a solution consisting of or consisting essentially of water and dissolved sodium chloride and dissolved sulfate or phosphate salt or acid).

The terminally sterilized intranasal baclofen formulation may have any suitable concentration of baclofen, such as between 0.01 mg/mL and 10 mg/mL baclofen. In embodiments, the terminally sterilized intranasal baclofen formulation has between 0.05 mg/mL and 2 mg/mL baclofen.

The pH may be adjusted with any suitable acid or base. In embodiments, the pH is adjusted with HCl, H₂SO₄, H₃PO₄ or NaOH.

Preferably, a therapeutically effective amount of baclofen is administered to a patient in need of treatment. By “therapeutically effective amount” herein is meant a dose that produces the effects for which it is administered. The exact dose will depend on the purpose of the treatment, and will be ascertainable by one skilled in the art using known techniques. In some embodiments, dosages of about 0.5 micrograms/kg to about 5 micrograms/kg are used. As is known in the art, adjustments for systemic versus localized delivery, and rate of new protease synthesis, as well as the age, body weight, general health, gender, diet, time of administration, drug interaction and the severity of the condition may be necessary, and will be ascertainable with routine experimentation by those skilled in the art.

In some embodiments, baclofen is delivered to a patient in a daily dose of between about 0.001 mg/kg/day to 100 mg/kg/day. A. “patient” for the purposes of the present disclosure includes both humans and other animals, particularly mammals including mice, rats, guinea pigs, rabbits, dogs, cats, swine, bovine, monkey, baboon, chimpanzee, and other organisms. Thus, the methods are applicable to both human therapy and veterinary applications. In a preferred embodiment the patient is a mammal, such as a human. Those “in need of treatment” include mammals already having the disease or disorder, as well as those prone to having the disease or disorder, including those in which the disease or disorder is to be prevented.

The baclofen formulations of the present invention allows for subsequent dilution by the addition of other components that are to be simultaneously intranasally administered with the baclofen. These include pain relieving medications suitable for combination with the baclofen and include morphine, clonidine, hydromorphine, hydrocodone, merperidine, celeroxib, tramadol, oxycodone, acetaminophen, ketoprofen, ketorolac, ibuprofen, naproxen, or the like. It is appreciated in the art that other chemical compounds are similarly suitable for co-administration or separate administration with baclofen in the current application.

In some embodiments, the pharmaceutical solution comprises at least one additional ingredient selected from the group consisting of: active pharmaceutical ingredients; enhancers; excipients; and agents used to adjust the pH, buffer the composition, prevent degradation, and improve appearance, odor, or taste.

### Carrier System

In some embodiments, the carrier system comprises one or more alcohols or glycols, or any combinations thereof, in an amount from about 10% to about 55%, about 10% to about 40%, about 10% to about 35%, about 12% to about 55%, about 12% to about 40%, about 12% to about 35%, about 15% to about 55%, about 15% to about 40%, about 15% to about 35%, about 10%, about 12.5%, about 15%, about 17.5%, about 20%, about 22.5%, about 25%, about 27.5%, about 30%, about 32.5%, about 35%, about 37.5%, about 40%, about 42.5%, about 45%, about 47.5%, about 50%, about 52.5% or about 55% (w/w). In some embodiments, the carrier system comprises one or more alcohols or glycols, or any combinations thereof, in an amount from about 25% to about 40% (w/w). In some embodiments, the carrier system comprises one or more alcohols or glycols, or any combinations thereof, in an amount of about 30% (w/w). In some embodiments, the alcohol is ethanol or contains ethanol. In some preferred embodiments, the glycols exclude glycol polymers. In some preferred embodiments, the glycols exclude glycol polymers having an average molecular weight of greater than 200. In some embodiments, the glycols exclude polyethylene glycol having an average molecular weight of greater than about 200.

Lower alkyl alcohols are those with six or fewer carbon atoms. Thus, any of ethanol, propyl alcohol, butyl alcohol, pentanol, benzyl alcohol, any isomers thereof, or any combinations thereof can be used.

Lower alkyl glycols are those with six or fewer carbon atoms. Thus, any of ethylene glycol, propylene glycol, butylene glycol, pentylene glycol, any isomers thereof, or any combinations thereof can be used.

### Additional Excipients

In some embodiments, a pharmaceutical solution comprises at least one penetration enhancer in addition to a baclofen. In some embodiments, the penetration enhancer is at least one alkyl glycoside. In some embodiments, the alkyl glycoside refers to any sugar joined to any hydrophobic alkyl, as described in U.S. Pat. No. 5,661,130, which is incorporated herein by reference in its entirety. The hydrophobic alkyl can be any suitable length, for example about 9 to about 24 carbons in length, especially about 10 to about 14 carbons in length. The hydrophobic alkyl can be branched and/or partially or wholly unsaturated. The alkyl may be joined to the saccharide core for example through a carbonyl group, whereby an ester group may be formed. A suitable alkyl glycoside will have the characteristics of being nontoxic, nonionic, and capable of increasing the absorption of a baclofen when it is administered intranasally as described herein. Exemplary saccharides that may be covalently joined to an alkyl according to the present invention include glucose, maltose, maltotriose, maltotetrose, sucrose and trehalose. Exemplary alkyl glycosides that may be employed include octyl-, nonyl-, decyl-, undecyl-, dodecyl, tridecyl, tetradecyl, pentadecyl, octadecyl .alpha.- or .beta.-D-maltoside,-glucoside or sucroside. In some embodiments, the preferred glycosides include maltose, sucrose or glucose linked by glycosidic linkage to an alkyl chain of 9, 10, 12, 14, 16, 18 or 20 carbon atoms. Specific excipients that may be employed in a nasal composition according to the invention include alkylsaccharide is dodecyl maltoside, tetradecyl maltoside, sucrose dodecanoate, sucrose monostearate, sucrose distearate, and/or combinations of two or more thereof. Where present, the amount of alkyl glycoside in the composition is sufficient to enhance the absorption of a baclofen administered by the intranasal route. In some embodiments, the amount of alkyl glycoside in the composition is selected so as to enhance absorption of the baclofen, while at the same time not significantly irritating the nasal mucosa. In some embodiments, the amount of alkyl glycoside in the composition is in a range of about 0.01% (w/v) to about 1% (w/v). In some embodiments, the amount of alkyl glycoside in the composition is in a range of about 0.05% (w/v) to about 0.5% (w/v), or about 0.125% (w/v) to about 0.5% (w/v).

The term “penetration enhancer”, means any material which acts to increase absorption across the mucosa and/or increases bioavailability. In some embodiments, such materials include mucolytic agents, degradative enzyme inhibitors and compounds which increase permeability of the mucosal cell membranes. Whether a given compound is an “enhancer” can be determined by comparing two formulations comprising a non-associated, small polar molecule as the drug, with or without the enhancer, in an in vivo or good model test and determining whether the uptake of the drug is enhanced to a clinically significant degree. The enhancer should not produce any problems in terms of chronic toxicity because in vivo the enhancer should be non-irritant and/or rapidly metabolized to a normal cell constituent that does not have any significant irritant effect.

In some embodiments, preferred enhancing materials lysophospholipids, for example lysophosphatidylcholine obtainable from egg or soy lecithin. Other lysophosphatidylcholines that have different acyl groups as well as lyso compounds produced from phosphatidylethanolamines and phosphatidic acid which have similar membrane modifying properties may be used. Acyl carnitines (e.g. palmitoyl-dl-carnitine-chloride) is an alternative. In some embodiments, a suitable concentration is from 0.02 to 20% (w/v).

In some embodiments, enhancing agents that are appropriate include chelating agents (EGTA, EDTA, alginates), surface active agents (especially non-ionic materials), acyl glycerols, fatty acids and salts, tyloxapol and biological detergents. Also agents that modify the membrane fluidity and permeability are appropriate such as enamines (e.g. phenylalanine enamine of ethylacetoacetate), malonates (e.g. diethyleneoxymethylene malonate), salicylates, bile salts and analogues and fusidates. Suitable concentrations are up to 20% (w/v).

### Mucosal Membrane Preparations

Mucosal membrane preparations are generally administered in metered sprays having volumes of less than 250 μL, preferably less than 150 μL, and ideally from 25 to 100 μL. Although not prohibited in this invention, administration of volumes larger than about 300 μL per dose usually exceeds the absorption capacity of the membranes. This results in a large portion of the pharmaceutically active ingredient being lost.

The dosage volume of preparations, in particular nasal preparations, preferably ranges from 25 to 100 μL. Volumes in excess of the aforementioned ranges may bypass the sinuses and flow down the back of the throat where the excess is swallowed.

### Administration

In some embodiments, the administration of the composition comprises administering at least a portion of the therapeutically effective amount of the baclofen onto at least one mucosal membrane. In some embodiments, the administration of the composition comprises spraying at least a portion of the therapeutically effective amount of the baclofen into at least one nostril. In some embodiments, the administration of the baclofen comprises spraying at least a portion of the therapeutically effective amount of the baclofen into each nostril. In some embodiments, the administration of the baclofen comprises spraying a first quantity of the baclofen into the first nostril, spraying a second quantity of the baclofen into a second nostril, and optionally after a pre-selected time delay, spraying a third quantity of the baclofen into the first nostril. Some embodiments further comprise, optionally after a pre-selected time delay, administering at least a fourth quantity of the baclofen to the second nostril.

### EXAMPLES

Aspects of the present teachings may be further understood in light of the following examples, which should not be construed as limiting the scope of the present teachings in any way.

### Example 1—Intranasal Baclofen Study

Forty (40) hospitalized patients with new acquired brain injury acutely treated in the PICU have been treated with intranasal baclofen. Aqueous intranasal solutions comprising 250 mcg, 1000 mcg, and/or 2000 mcg of baclofen and 9 grams of sodium chloride per mL exploiting the nasal pathway have delivered baclofen to the CNS by circumventing the BBB.

The response to intranasal baclofen is far greater than that observed with enteral baclofen even when enteral baclofen dose has been maximized (>90 mg/day). Intranasal baclofen has been used for patients with new acquired brain injuries suffering from hypertonicity or paroxysmal sympathetic hyperactivity (PSH) where historically enteral baclofen has been the first line mediation. The efficacy of intranasal baclofen has exceeded the effects observed with enteral baclofen with far less sedation.

### Example 2—Individual Intranasal Baclofen Treatment

A patient was admitted to a pediatric intensive care unit (PICU) due to increased tone after the patient had underwent surgical explantation of an intrathecal baclofen pump. The patient was on maximum doses of several enteral medications including enteral baclofen. He was given a test dose of intranasal baclofen administered in the PICU using commercially prepared intrathecal baclofen for test dose (gablofen 2-50 mcg vials delivered intranasally as one administration). Significant improvement in tone was noted despite all enteral medications being held before intranasal challenge.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Specifically intended to be within the scope of the present invention, and incorporated herein by reference in its entirety, are the following publications:
1. Pandyan A D, Gregoric M, Barnes M P, et al. Spasticity: clinical perceptions, neurological realities and meaningful measurement. Disabil Rehabil 2005;27:2-6.
2. Sanger T D, Delgado M R, Gaebler-Spira D, et al. Classification and definition of disorders causing hypertonia in childhood. Pediatrics. 2003; 111(1):e89-e97.
3. Tilton A H. Therapeutic interventions for tone abnormalities in cerebral palsy. The American Society for Experimental NeuroTherapeutics 2006;3:217-24.
4. Hudgson P, Weightman D. Baclofen in the treatment of spasticity. Br Med J. 1971;4(5778):15-27.
5. Knutsson E, Lindblom UL, Mårtensson A. Plasma and cerebrospinal fluid levels of baclofen (Lioresal®) at optimal therapeutic responses in spastic paresis. Journal of the neurological sciences. 1974 Nov. 1;23(3):473-84.
6. Zhang H, Schmidt M, Murry D J, Donovan M D. Permeation and systemic absorption of R- and Sbaclofen across the nasal mucosa. J Pharm Sci. 2011; 100(7):2717-2723. doi:10.1002/jps.22499
7. Penn R D, Kroin J S. Continuous intrathecal baclofen for severe spasticity. Lancet. 1985;2(8447):125-127.
8. Ramstad K, Jahnsen R, Lofterod B, Skjeldal O H. Continuous intrathecal baclofen therapy in children with cerebral palsy—when does improvement emerge? Acta Paediatric. 2010; 99:1661-65.
9. Kraus T, Gegenleitner K, Svehlik M, Novak M, Steinwender G, Singer G. Long-term therapy with intrathecal baclofen improves quality of life in children with severe spastic cerebral palsy. Eur J Paediatr Neurol 2017; 21:565-69.
10. Dan B, Motta F, Vles J S, et al. Consensus on the appropriate use of intrathecal baclofen (ITB) therapy in paediatric spasticity. Eur J Paediatr Neurol 2010; 14(1): 19-28.
11. Imerci A, Rogers K J, Pargas C, Sees J P, Miller F. Identification of Complications in Paediatric Cerebral Palsy Treated with Intrathecal Baclofen Pump: A Descriptive Analysis of 15 Years at One Institution. J Child Orthop 2019; 13(5): 529-535.
12. Haranhalli N, Anand D, Wisoff J H, Harter D H, Weiner H L, Blate M, Roth J. Intrathecal baclofen therapy: complication avoidance and management. Child's Nervous System. 2011 Mar;27(3):421-7.
13. Ghadiri M, Young P M, Traini D. Strategies to Enhance Drug Absorption via Nasal and Pulmonary Routes. Pharmaceutics. 2019 Mar 11;11(3):113. DOI handle “10.3390/pharmaceutics11030113”. PMID: 30861990; PMCID: PMC6470976
14. Agu R. U., Ugwoke M. I., Armand M., Kinget R., Verbeke N. The lung as a route for systemic delivery of therapeutic proteins and peptides. Respir. Res. 2001;2:198-209.

1. A method of treating a patient with a disorder that may be treatable with a baclofen, the method comprising:
administering to one or more nasal mucosal membranes of a patient a pharmaceutically acceptable solution for nasal administration consisting of a baclofen in a saline solution.

2. The method of claim 1, wherein the disorder is selected from the group consisting of muscle hypertonicity, spasticity, and dystonia.

3. The method of claim 1, wherein the saline solution further comprises an additive selected from the group consisting of citric acid, cyclodextrin, and hypromellose.

4. The method of claim 1, wherein the pharmaceutical solution is in a pharmaceutically acceptable spray formulation having volume from about 10 μL to about 200 μL.

5. The method of claim 1, wherein the administration of the pharmaceutical solution comprises spraying at least a portion of the therapeutically effective amount of the baclofen into at least one nostril.

6. The method of claim 1, wherein the administration of the pharmaceutical solution comprises spraying at least a portion of the therapeutically effective amount of the baclofen into each nostril.

7. The method of claim 1, wherein the administration of the pharmaceutical solution comprises spraying a first quantity of the pharmaceutical solution into the first nostril, spraying a second quantity of the pharmaceutical solution into a second nostril, and optionally after a pre-selected time delay, spraying a third quantity of the pharmaceutical solution into the first nostril.

8. The method of claim 7, further comprising, optionally after a pre-selected time delay, administering at least a fourth quantity of the pharmaceutical solution to the second nostril.

9. The method of claim 1, wherein nasal administration of the pharmaceutical solution begins at any time before or after onset of symptoms of a disorder which may be treatable with the pharmaceutical solution.

10. The method of claim 1, wherein the treatment achieves bioavailability that is from about 80-125% of that achieved with the same baclofen administered intrathecally.

11. A pharmaceutical solution for nasal administration consisting of:
(a) a baclofen or its pharmaceutically acceptable salt; and
(b) one or more of citric acid, cyclodextrin, and hypromellose, or any combinations thereof, in an amount from about 30% to about 95% (w/w); in a pharmaceutically acceptable formulation for administration to one or more nasal mucosal membranes of a patient.

12. The pharmaceutical solution of claim 11, wherein the baclofen is present in the pharmaceutical solution in a concentration from about 2.0 mg/mL to about 10.0 mg/mL.

13. A device for delivery of a pharmaceutical solution, the device comprising:
the pharmaceutical solution of claim 11, wherein the pharmaceutical solution is stored in a cartridge;
an atomizer, wherein the atomizer receives the pharmaceutical formulation from the cartridge and delivers the baclofen formulation to a patient's nasal cavity;
an optional dose adjustment control, wherein the dose adjustment control determines the amount of the pharmaceutical formulation to be delivered; and
a spray control, wherein the spray control triggers the pharmaceutical formulation to be delivered to the patient's nasal cavity.

14. The method of claim 1, wherein the baclofen comprises R-baclofen (D baclofen), S-baclofen (L-baclofen), or mixtures thereof.

15. The method of claim 1, wherein the baclofen comprises a racemate comprising a mixture of R-and S-baclofen (DL-baclofen) in equal proportions.

16. The method of claim 1, wherein the baclofen is R-baclofen.

17. The pharmaceutical solution of claim 11, wherein the baclofen comprises R-baclofen (D baclofen), S-baclofen (L-baclofen), or mixtures thereof.

18. The pharmaceutical solution of claim 11, wherein the baclofen comprises a racemate comprising a mixture of R-and S-baclofen (DL-baclofen) in equal proportions.

19. The pharmaceutical solution of claim 11, wherein the baclofen is R-baclofen.

20. The device of claim 13, wherein the baclofen comprises R-baclofen (D baclofen), S-baclofen (L-baclofen), or mixtures thereof.
